(19) United States
(12) Patent Application Publication
(10) Pub. No.: US 2007/0021684 A1
(43) Pub. Date: Jan. 25, 2007

(54) Tissue punching device

(76) Inventors: Markus Brielmeier, Garching (DE); Joerg Schmidt, Munich (DE)

Correspondence Address: JENKINS, WILSON, TAYLOR & HUNT, P. A., 3100 TOWER BLVD, SUITE 1200, DURHAM, NC, 27707, US

(21) Appl. No.: 10/548,729

(22) PCT Filed: Mar. 10, 2004
(86) PCT No.: PCT/EP04/02457

§ 371 Date: May 30, 2006

(30) Foreign Application Priority Data
Mar. 25, 2003 (DE) 103 13 340.2

(51) Int. Cl. A61B 10/00; A61B 17/32

(52) U.S. Cl. 600/564; 606/184

(57) ABSTRACT

The device for punching out a tissue sample comprises a receiving container for receiving the tissue sample, with a container closure element which can be inserted into the receiving container in order to close said receiving container, the container closure element having a punch aperture, and with a clip which is connected to the container closure element and carries a punch that can be lowered with an exact fit into the punch aperture.

The invention relates to a device for punching out a tissue sample, in particular from animals, for the purpose of identifying or marking them and for determining their genotype.

In medical research, increasing use is being made of genetically altered animals, also referred to as transgenic animals. When breeding these animals for research purposes, the genotype of each newborn animal is determined so that the desired characteristics are recognized and are used for future breeding. This is usually done by analysis of tissue samples by molecular biological methods, for example the polymerase chain reaction (PCR). To obtain the tissue sample, a piece of tail is usually cut off, generally without anaesthesia, from the animal that is to be genotyped, for example a mouse. After incubation of the tissue sample in an enzyme solution and subsequent DNA isolation, a characteristic gene segment of this sample is copied and analyzed by means of molecular biological methods, mainly the polymerase chain reaction (PCR).

The molecular biological methods for obtaining a characteristic gene segment of the tissue sample are very sensitive and are specific to the transgene that is to be investigated. However, because of their sensitivity, they are extremely susceptible to even the very slightest contamination from tissues of other animals. If a tissue sample is first removed from a transgenic positive animal and then a tissue sample is taken from an animal that is non-transgenic, there is a danger that, by using the same device to collect samples, transgenic positive tissue sections will be transferred and the subsequent non-transgenic animal will be erroneously identified as being transgenic.

DE 197 40 429 A1 describes a method and a device for collection of biological samples. By means of forceps, a sample capsule closure element with a sharp outer edge is pressed in the manner of a stamp through the tissue and against the sample capsule container acting as die, so that a tissue sample is cut off in this way. By a round groove in the sample capsule container and the corresponding mating piece in the sample capsule closure element, both parts are closed together to form a sample capsule, such that undesired opening is avoided. DE 197 40 429 A1 also describes a sample capsule in which the sample container and the associated lid are in one part and are connected to one another via a hinge.

EP 1 088 212 B1 describes a device and a method for collection and initial preparation of tissue samples for molecular genetic diagnosis. With the aid of ear forceps, a sample collection device with a sharp-edged front end is pushed through the ear and, in the same operating step, pressed into the sample-receiving container. The sample collection device is fixed in the sample-receiving container by a fastening means.

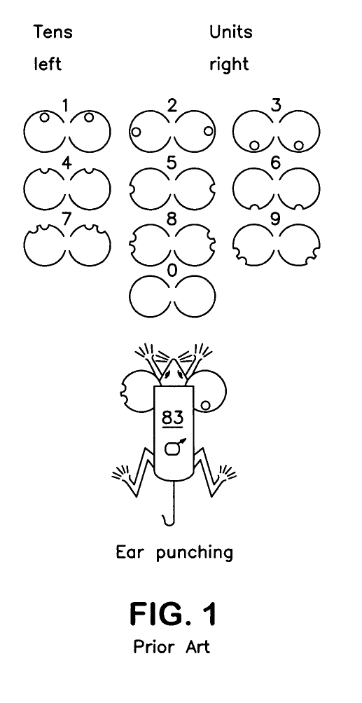

To ensure that the tissue samples can be assigned to the correct animals, the animals to be investigated have to be marked. This is usually done by punching the ears, i.e. the ears of the animal to be investigated are marked by suitable punching. FIG. 1 shows an example of the coding used to identify the animal to be investigated. Each number is assigned a corresponding punch pattern, as is shown in FIG. 1. For example, the number 1 is represented by a round punch at the front margin of the ear, the number 2 by a punch at the side of the ear, and the number 3 by a punch at the rear margin of the ear. The numbers 4 to 6 are correspondingly represented by notches or semicircular punches. The numbers 7 to 9 are represented by double punches or double notches. If the ear is unpunched, this stands for the number 0. By convention, the left ear is used to indicate multiples of the number 10, and the right ear to indicate numerical values from 1 to 9. In the example shown in FIG. 1, therefore, this is the 83rd animal of the group to be investigated.

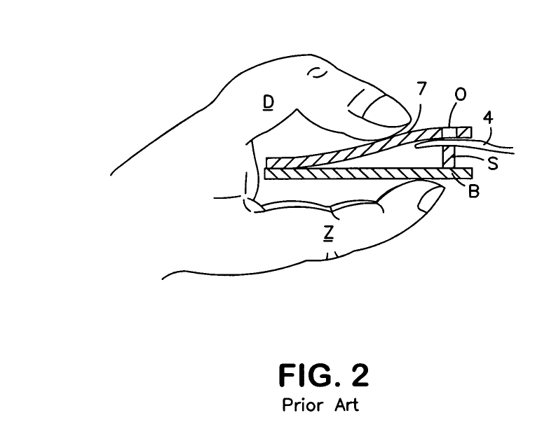

FIG. 2 shows a conventional device for marking mice in accordance with the coding plan indicated in FIG. 1. The punching device is made up of a base plate B on which a cylindrical punching means S is arranged. The base plate also carries a metal leaf spring F in which a punch aperture O is provided. The punch spring F can be lowered by manual pressure, the punch S being inserted quite precisely into the punch aperture O. When marking the mouse, the left or right mouse ear M is pushed between the punch spring F and the punch cylinder S, and the punch spring F is then pressed down manually so that a tissue section of the mouse ear M is punched out through the aperture O. The punching device shown in FIG. 2 and used for marking mice according to the prior art is made of a metal which can be easily sterilized by heat. By using metal, it is possible for the punching device shown in FIG. 2 to be used for a large number of marking operations. Alternatively, the punching means S can also be arranged on the leaf spring, and the punch aperture O in the base plate.

The punching device shown in FIG. 2 and used for marking ears is not suitable for punching out tissue samples. Since the ears of the animals to be investigated have a predetermined size, the punched-out markings likewise cannot exceed a certain size. The aperture O in the conventional marking device shown in FIG. 2 has a diameter of about two millimetres. Accordingly, the tissue section punched out from the ear M is also relatively small and has a maximum diameter of two millimetres. To actuate the marking device according to FIG. 2, it is usually gripped between a thumb D and index finger Z of the operator. With appropriate handling of the marking device, the punched-out tissue section drops upwards, or alternatively downwards, through the aperture O and can be collected in a receiving container for further analysis. However, the actuation of the marking device for this purpose is very awkward, since the marking device according to FIG. 2 is operated with one hand while the mouse whose ear is to be marked has to be held with the other hand. It is therefore not generally possible for the operator to collect the punched-out tissue sample for further analysis in a receiving vessel located underneath it.

A further serious disadvantage of conventional punching devices is that the marking device shown in FIG. 2 for marking mice is a reusable instrument, i.e. the punching device is used for marking a large number of different animals to be investigated. It may therefore happen that tissue residues or blood cells remain attached to the punch S or in the aperture O of the marking device and thus falsify the result of the examination of the subsequently marked animals.

The object of the present invention is therefore to make available a device for punching out a tissue sample, in which the punched-out tissue sample is safely collected in a test vessel, and test results are not falsified by transfer of tissue samples.

According to the invention, this object is achieved by a device having the features set out in Patent claim 1.

The invention makes available a device for punching out a tissue sample, with
a receiving container for receiving the tissue sample,
a container closure element which can be inserted into the receiving container in order to close said receiving container,
the container closure element having a punch aperture, and with a clip which is connected to the container closure element and carries a punch that can be lowered with an exact fit into the punch aperture.

The underlying idea of the invention is to make available a device in which, in one operating step, both a marking of the animal to be investigated takes place and, at the same time, an associated tissue sample is taken from the animal.

This has the particular advantage of entailing one less operating step and, in addition, incorrect allocation between the marked animal and its removed tissue sample is largely eliminated.

A further advantage of the device according to the invention is that, besides the marking in the ear, it is not additionally necessary to remove the tip of the animal's tail by amputation. This represents a contribution to the welfare of the animals.

The device according to the invention for punching out a tissue sample is a disposable instrument, i.e. the punching device according to the invention is used on a single test animal.

In a preferred embodiment of the device according to the invention, the container closure element and the clip are made of plastic.

The punch is preferably arranged at a distal end of the clip and, by application of mechanical pressure, can be lowered into the punch aperture of the container closure element counter to an elastic force of the clip.

In a particularly preferred embodiment, a holding means is additionally provided in which the receiving container closed by the container closure element is suspended.

This holding means preferably comprises a manually actuated compression spring via which pressure can be applied mechanically to the clip.

An advantage of the holding means is that it greatly facilitates manual actuation of the punching device according to the invention.

In a preferred embodiment of the device according to the invention, the punch carried by the clip and lowered with an exact fit into the punch aperture springs back from the container closure element, by means of the elastic force of the clip, for renewed actuation if the mechanical pressure applied manually via the compression spring is low.

In a preferred embodiment of the punching device according to the invention, the punch carried by the clip and lowered with an exact fit into the punch aperture remains in the container closure element, in order to seal the latter, if the mechanical pressure applied manually via the compression spring is terminated only after a longer time, or if greater pressure is applied when punching the last sample from an animal.

The punch carried by the clip is preferably connected to the clip via a predetermined breaking point.

In a preferred embodiment, the spring of the holding means is made of metal.

In an alternative embodiment, the spring of the holding means is made of plastic.

The receiving container of the punching device according to the invention is preferably cylindrical, the punch aperture of the container closure element being arranged eccentrically with respect to the axis of symmetry of the receiving container.

The receiving container is preferably transparent.

In a particularly preferred embodiment of the punching device according to the invention, a reaction fluid for the punched-out tissue sample has already been introduced into the receiving container.

This has the particular advantage that the operating step involving introducing the reaction fluid after the punching procedure is no longer necessary.

In a particularly preferred embodiment of the punching device according to the invention, the receiving container is an Eppendorf tube or a vessel similar to this.

Preferred embodiments of the device according to the invention for punching out a tissue sample are described below with reference to the attached figures in order to explain the features essential to the invention.

FIG. 1 shows a coding scheme for marking test animals in accordance with the prior art;

FIG. 2 shows a marking device for marking test animals in accordance with the prior art;

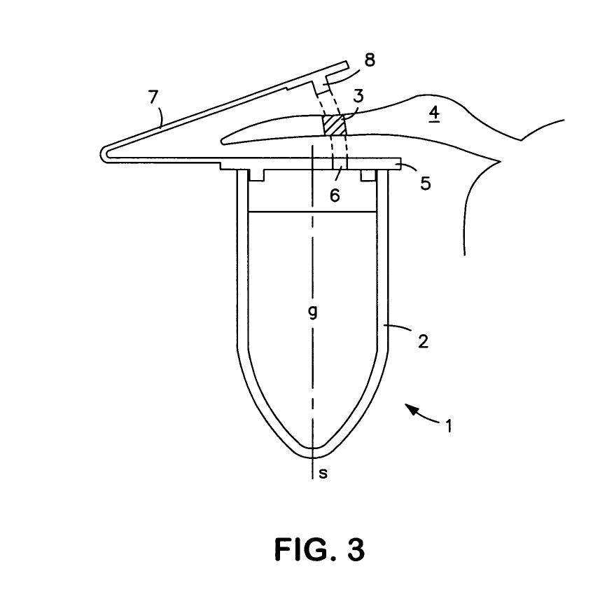

FIG. 3 shows a first embodiment of the device according to the invention for punching out a tissue sample;

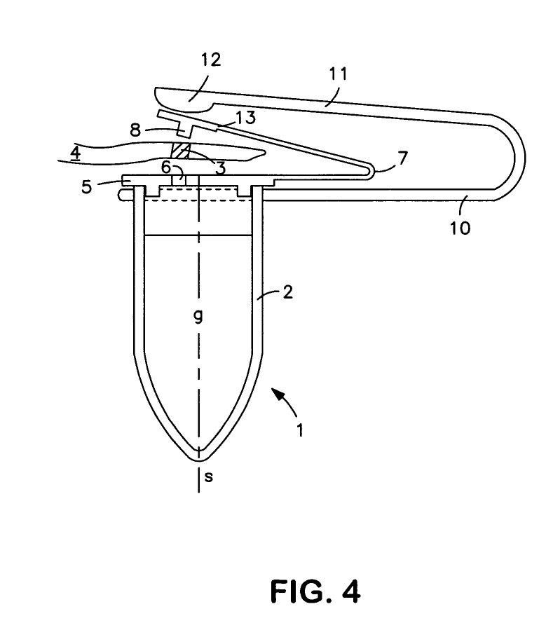

FIG. 4 shows a preferred embodiment of the device according to the invention for punching out a tissue sample;

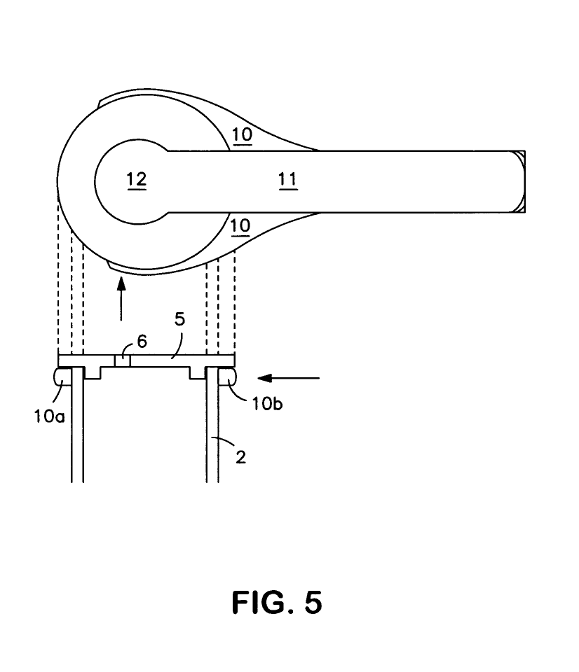

FIG. 5 shows a top view of the punching device depicted in FIG. 4; and

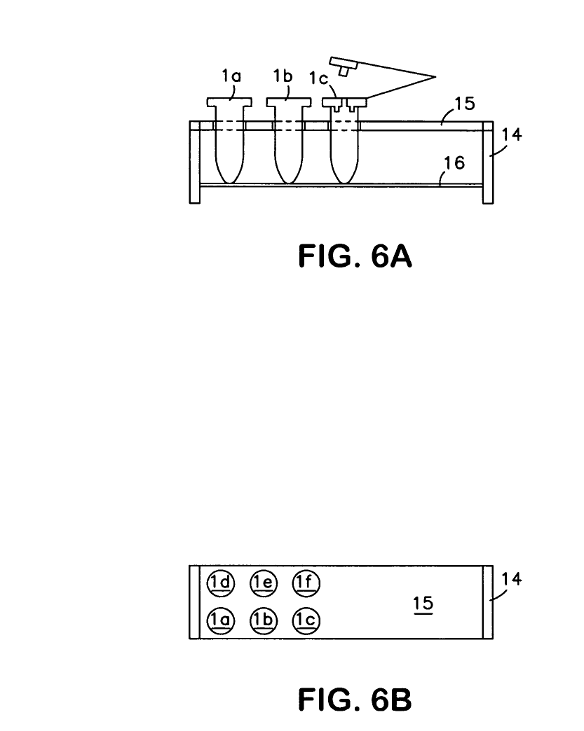

FIGS. 6a and 6b show a holder device for holding several punching devices according to the invention.

FIG. 3 shows a preferred embodiment of a punching device 1 according to the invention for punching out a tissue sample. The punching device 1 according to the invention comprises a receiving container 2. The receiving container 2 is used for receiving a tissue sample 3 punched out from an ear 4 of a test animal. The receiving container 2 is open at the top, and a container closure element 5 can be inserted into the opening in order to close the receiving container 2. The container closure element 5 is connected releasably to the receiving container 2. For example, the container closure element 5 is pressed into the opening of the receiving container 2. In a particular embodiment, the container closure element 5 snaps into the receiving container 2. The receiving container 2 is preferably an Eppendorf tube.

In a particularly preferred embodiment, a reaction fluid for the punched-out tissue sample has already been introduced into the receiving container 2. This reaction fluid is preferably an enzyme solution for DNA isolation.

The container closure element 5 has a punch aperture 6, which is preferably round. The receiving container 2 shown in FIG. 3 is preferably cylindrical and symmetrical with respect to a line of symmetry S. The punch aperture 6 preferably does not lie on the line of symmetry S, but instead eccentrically with respect to the latter, as is shown in FIG. 3. In this way, it is possible also to apply punched holes at the rear end of the ear 4.

On the container closure element 5 there is a clip 7 which carries a punch 8 that can be lowered with an exact fit into the punch aperture 6. The container closure element 5 and the clip 7 connected integrally to it are preferably made of plastic, in particular of polystyrene. The diameter of the punch aperture 6 and of the cylindrical punch 8 is preferably 2 mm±0.5 mm. In this way, the punching device 1 is suitable for marking mouse or rat ears in accordance with FIG. 1. By virtue of the relatively small diameter of the punch aperture 6, the reaction fluid 9 contained in the receiving container 2 does not flow out of the receiving container 2 on account of its surface tension, even if the punching device 1, during punching-out of the tissue samples, is held in such a way that the punch aperture 6 points downward. In a first embodiment of the punching device 1 according to the invention, the container closure element 5 and clip 7 form a first structural component, which can be inserted into the receiving container 2 as separate structural component. This allows the operator to introduce a reaction fluid 9 suitable for his specific research purposes into the receiving container 2 and then to punch out the tissue sample.

In an alternative embodiment, the container closure element 5 and its clip 7 are connected fixedly to the receiving container 2 after the receiving container 2 has been filled with a predetermined reaction fluid. In this embodiment, the reaction fluid has already been introduced into the receiving container 2 by the manufacturer and delivered to the customer. For the operator, this has the advantage that the reaction fluid does not still have to be introduced manually into the receiving container 2. The container closure element is preferably delivered in a closed state, with the clip 7 pressed down, i.e. the punch 8 closes the punch aperture 6. This configuration affords the particular advantage that the reaction fluid 9 introduced into the receiving container 2 is protected from contamination.

To obtain a tissue sample, the operator takes the punching device 1 in his hand and grips the V-shaped clip 7 between two fingers. The ear 4 of the test animal is then guided between the two branches of the clip 7, and the punch 8 is pressed manually into the punch aperture 6. The cylindrical punching means 8 preferably has sharp edges which cut into the tissue 4 so that a cylindrical tissue sample 3 drops into the receiving container 2. After incubation of the tissue section 3 in the enzyme solution 9 and subsequent DNA isolation, molecular biological methods, for example PCR, are used to copy a characteristic gene segment from the tissue sample 3 and then analyze it. To do this, a liquid sample is preferably pipetted out of the receiving container 2.

FIG. 4 shows a particularly preferred embodiment for better, safer and more sensitive handling of the punching device 1 according to the invention. In the embodiment shown in FIG. 4, the punching device 1 additionally comprises a holding means 10 into which the receiving container 2 closed by the container closure element 5 is suspended. This holding means 10 is preferably made of metal or of plastic. The holding means 10 has a manually actuated compression spring 11 whose endpiece 12 can be pressed manually against the end of the clip 7. The receiving container 2 is preferably what is called an Eppendorf tube. These Eppendorf tubes are usually relatively small in terms of their geometric dimensions. The holding means 10 is configured in such a way that it has a size permitting straightforward handling by an operator. The operator applies a pressure force on the spring 11. This force is transmitted by the bulge 12 to the punch 8, which punches out the tissue section 3 from the ear. The punched-out tissue section 3 drops through the aperture 6 into the reaction fluid 9 in the receiving container 2. The spring 11 is preferably made of metal. In an alternative embodiment, the spring 11 is made of plastic.

A particular advantage of the embodiment shown in FIG. 4 is that there is no contact between the operator's hand and the tissue section 3. This ensures that residues of materials present on the operator's hand do not contaminate the tissue section 3.

In a preferred embodiment of the punching device 1 according to the invention, the punch 8 carried by the clip 7 and lowered with an exact fit into the punch aperture 6 is sprung back by the elastic force of the clip 7 for renewed actuation after the punching operation. This occurs especially if the mechanical pressure applied manually via the compression spring 11 is low. If the operator presses the compression spring 11 for a longer time or with greater pressure against the lowered clip 7, in a preferred embodiment, the lowered punch 8 remains in the container closure element 5 and seals the latter. In this case, the clip 7 in a particularly preferred embodiment has a predetermined breaking point 13.

For example, if a tissue sample is taken from the mouse 83 in FIG. 1 using the punching device 1 according to the invention, this mouse is to be marked by three punching operations. In this punching operation, an eight is to be marked on the left ear and a three on the right ear. For this purpose, the punching device 1 according to the invention is initially applied twice to the margin of the left ear, and two semicircular punches or notches are formed. The right ear is then marked as a three using the punching device 1 according to the invention. The three punching operations can be carried out in any desired sequence. In the third and last punching operation, the operator presses the spring 11 against the punch 8 for such a length of time or with such a pressure that the punch 8 remains fixed in the punch aperture 6 and completely closes the receiving container 2. In the first and second punching operations, the operator applies a pressure to the spring 11 such that the clip 7 can spring back for further actuation. Only in the third punching operation does the predetermined breaking point 13 break, and the punch 8 remains fixed in the container closure element. A new receiving container 2 is then set up for marking the next animal.

From the test animal 83 shown in FIG. 1, three tissue sections are punched out and drop into the reaction fluid 9. The marking procedure and the recovery of a tissue sample from a test animal take place in one operating step according to the invention. In the device according to the invention, the reaction fluid 9 can also be introduced into the receiving container 2 by the manufacturer and made available in this state for the experiment. The reaction fluid is introduced by the operator in the laboratory according to his particular requirements, before he enters the room containing the test animals.

The receiving container 2 is preferably transparent, so that the operator can check whether it contains the desired enzyme solution and whether the removed tissue sample 3 has dropped into the reaction fluid 9. After the tissue sample 3 has dropped into the receiving container 2, the associated number of the test animal is written by pen on the receiving container 2. In the example shown in FIG. 1, for example, the operator writes the number 83 on the receiving container 2 using a pen.

In FIG. 5, the punching device in FIG. 4 is shown from above and in a front cross section.

FIGS. 6a and 6b show a stand 14 for holding several punching devices 1 according to the invention. FIG. 6a shows a cross-sectional view, and FIG. 6b shows a view from above. In the example shown in FIG. 6, six punching devices 1 according to the invention from FIG. 3 are held in the stand 14. The stand 14 has a first rack 15 in which there are openings to hold the receiving containers 2. A second rack 16 holds the tips of the receiving containers 2.

The punching device 1 according to the invention is suitable for punching out tissue samples from any desired organisms or animals. The test animals are in particular mice, rats or fish. In the case of fish, the punching device 1 according to the invention is used to punch a fin of the fish to be examined. Since the punching device 1 according to the invention is preferably a disposable instrument, it is also possible to dispense with the otherwise customary cleaning of the sample collection instrument. In the punching device 1 according to the invention, inadvertent transfer of tissue samples is eliminated. The punching device 1 according to the invention can be used both for the area of research and also for animal breeding. During the punching operation, the punched-out tissue sample does not come into contact with the operator's hand and contamination of the sample by contaminants present on the operator's hand is avoided in this way. The punching device 1 according to the invention is especially easy to handle and can be used in every position. Allocation of the tissue samples 3 to the test animals is made considerably easier because the marking procedure and the tissue sample collection take place in one operating step. Amputation of the end of the tail, toes or fins of test animals can be avoided, so that the punching device 1 according to the invention also serves to protect animals. Contamination of the reaction fluid introduced beforehand into the receiving container 2 can be avoided in every case.

### LIST OF REFERENCE NUMBERS

1 punching device
2 receiving container
3 punched-out tissue sample
4 ear
5 container closure element
6 punch aperture
7 clip
8 punch
9 reaction fluid
10 holding means
11 compression spring
12 endpiece with bulge
13 predetermined breaking point
14 stand
15 stand rack
16 stand rack

1. Device for punching out a tissue sample, with:
(a) a receiving container for receiving the tissue sample,
(b) a container closure element which can be inserted into the receiving container in order to close said receiving container, the container closure element having a punch aperture, and
(c) a clip which is connected to the container closure element and carries a punch that can be lowered with an exact fit into the punch aperture.

2. Device according to claim 1, wherein the container closure element and the clip are made of plastic.

3. Device according to claim 1, wherein the punch is arranged at the distal end of the clip and, by application of mechanical pressure, can be lowered into the punch aperture of the container closure element counter to an elastic force of the clip.

4. Device according to claim 1, wherein a holding means is provided in which the receiving container closed by the container closure element is suspended.

5. Device according to claim 4, a holding means comprises a manually actuated compression spring via which pressure can be applied mechanically to the clip.

6. Device according to claim 5, wherein the punch carried by the clip and lowered with an exact fit into the punch aperture springs back from the container closure element, by means of the elastic force of the clip, for renewed actuation if the mechanical pressure applied manually via the compression spring is terminated within a very short time.

7. Device according to claim 5, wherein the punch carried by the clip and lowered with an exact fit into the punch aperture remains in the container closure element, in order to seal the latter, if the mechanical pressure applied manually via the compression spring is terminated only after a longer time.

8. Device according to claim 7, wherein the punch carried by the clip is connected to the clip via a predetermined breaking point.

9. Device according to claim 5, wherein the compression spring of the holding means is made of metal.

10. Device according to claim 5, wherein the compression spring of the holding means is made of plastic.

11. Device according to claim 1, wherein the receiving container is cylindrical, the punch aperture of the container closure element being arranged eccentrically with respect to the axis of symmetry of the receiving container.

12. Device according to claim 1, wherein the receiving container is transparent.

13. Device according to claim 1, wherein a reaction fluid for the punched-out tissue sample is introduced into the receiving container.

14. Device according to claim 13, wherein the reaction fluid is an enzyme solution.

15. Device according to claim 1, wherein the receiving container is an Eppendorf tube.
